(19) United States
(12) Patent Application Publication
(10) Pub. No.: US 2021/0246404 A1
(43) Pub. Date: Aug. 12, 2021

(54) DEVICE AND METHOD FOR STORING LIVE MICROALGAE

(71) Applicant: Vaxa Technologies Ltd., Rosh Pina (IL)

(72) Inventors: Ohad BASHAN, Sde Varburg (IL); Isaac BERZIN, Jerusalem (IL); Oded BASHAN, Rosh Pina (IL); Sam C. COUTURE, Heartland, VT (US)

(73) Assignee: Vaxa Technologies Ltd., Rosh Pina (IL)

(21) Appl. No.: 17/052,564

(22) PCT Filed: May 2, 2019
(86) PCT No.: PCT/IL2019/050489

§ 371 Date: Nov. 3, 2020

Related U.S. Application Data
(60) Provisional application No. 62/666,117, filed on May 3, 2018.

(51) Int. Cl. C12M 1/00; C12M 1/02; C12M 1/34; C12N 1/12; C12N 1/04

(52) U.S. Cl. CPC C12M 21/02; C12M 23/22; C12M 41/06; C12M 27/00; C12M 45/22; C12M 41/14; C12M 41/32; C12N 1/12; C12N 1/04; C12M 41/34

(57) ABSTRACT

A device for storing live algae is disclosed. The device may include: a closed container, at least partially transparent to light, the container is configured to hold live algae aquaculture at a predetermined temperature; at least one light source for providing light to the closed container; a CO₂ source for providing CO₂ to the closed container; an air circulation system for circulating air inside the closed container; and a controller for controlling the at least one light source to illuminate an internal space of the closed container in an amount sufficient to keep the algae aquaculture alive but inhibits reproduction of the algae for at least 4 weeks.

### TECHNICAL FIELD OF THE INVENTION

This application relates to the field of storing algae and more specifically to devices and methods for storing live algae.

### BACKGROUND OF THE INVENTION

Many organisms being artificially grown in aquaculture is nourished with algae (either microalgae or macroalgae). Organisms, such as, finfish, molluscs and crustaceans such as seabass, seabream, abalone, bivalves, shrimps and the like require to be nourished with live algae at least in some stages in their life. Live algae have higher nutrition values than any other alternatives, such as, dried or frozen algae. Algae is either dried or frozen in order to extend the shelf life of the product and to allow the use of cheaper storage and/or shipment methods, such as sea shipping. Live microalgae may survive up to 4 weeks in aquaculture if kept cooled (e.g., in a refrigerator). Accordingly, distribution of live algae nowadays requires to use an expensive air shipping,

Some aquaculture organisms' hatcheries grow their own live algae/microalgae but, this process is limited to low algae densities (e.g., 0.5 g/L microalgae) and is complicated and expensive.

Accordingly, there is a need for a storage device and method that may allow to store live microalgae for periods of time extending 4 weeks, for example, 2 months, 6 months, 12 months and more.

### SUMMARY OF THE INVENTION

Some aspects of the invention may be related to a device for storing live algae. The device may include: a closed container, at least partially transparent to light, the container is configured to hold live algae aquaculture at a predetermined temperature; at least one light source for providing light to the closed container; a CO₂ source for providing CO₂ to the closed container; an air circulation system for circulating air inside the closed container; and a controller for controlling the at least one light source to illuminate an internal space of the closed container in an amount sufficient to keep the algae aquaculture alive but inhibits reproduction of the algae for at least 4 weeks.

In some embodiments, the controller may further be configured to control the CO₂ source to provide CO₂ in an amount sufficient to keep the algae aquaculture alive but inhibits reproduction of the algae. In some embodiments, the intensity and duration of the light may be determined based on at least one of: the type of live algae, the size of the closed container's internal space, and a required the live algae density. In some embodiments, the amount of CO₂ may be determined based on at least one of: the type of the live algae, the size of the closed container's internal space, and a required live algae density.

In some embodiments, the controller may be configured to adjust the predetermined temperature. In some embodiments, the device may further include a controlled temperature chamber adapted to hold the closed container. In some embodiments, the device may further include at least one sensor for sensing one or more conditions inside the closed container, and the controller may be configured to control one or more controllable components of the device based on the one or more sensed conditions.

In some embodiments, the device may further include an oxidative agent source. In some embodiments, the controller may further be configured to control (e.g., adjust, change or otherwise manipulate) the timing and amount of oxidative agent provided to the closed container from the oxidative agent source to be sufficient to prevent a grow of undesired microorganisms while not harming the live algae aquaculture. In some embodiments, the device may further include a humidifier for adding humidity to the circulated air. In some embodiments, the closed container may have a volume of 0.5-20 liters.

Some additional aspects of the invention may be directed to a method of controlling the population of live algae in a device for storing live algae. Embodiments of the method may include circulating air in algae aquaculture stored in a closed container included in the device; providing CO₂ to the algae aquaculture; and providing a controlled amount of light radiation to the algae aquaculture stored in the closed container. In some embodiments, the light is provided in amounts sufficient to keep the algae aquaculture alive while inhibiting reproduction of the algae in the aquaculture for more than 4 weeks.

Some embodiments of the method may further or alternatively include providing to the algae aquaculture a controlled amount of CO₂ sufficient to keep the algae aquaculture alive while inhibiting reproduction of the algae in the aquaculture. Some embodiments of the method may include controlling the temperature of the algae aquaculture to keep the algae aquaculture alive while inhibiting the reproduction of the algae in the aquaculture. Some embodiments of the method may include providing oxidative agent to the algae aquaculture. In some embodiments, providing the oxidative agent may include providing predetermined doses of oxidative agent at predetermined timing.

Some embodiments of the method may include providing humidity to the circulated air. In some embodiments, the amount of humidity in the air is determined such that an amount of water in the live algae aquaculture stored in the smart container is kept substantially constant.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is diagrammatic illustration of a device for storing live algae according to some embodiments of the invention; and

FIG. 2 is a flowchart of a method of storing live algae according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

A device and method according to some embodiments of the invention may allow to extend the shelf life of live algae in aquaculture (e.g., microalgae and macroalgae) for relatively long storing period (e.g., 12 months). A method and a device according to some embodiments may allow to use shipping (e.g., in sea) as the distribution method. In some embodiments, during storing the nutrition values of the algae may be maintained as the nutrition values of a freshly harvested algae. In some embodiments, keeping live algae in closed containers for long periods (e.g., during shipping or in storage) may require providing the algae with CO₂ and light in amounts sufficient to keep the algae aquaculture alive, but insufficient to allow the algae to reproduce. In such case the amount and density of the algae in the container may be kept substantially constant while preserving the vitality of the algae.

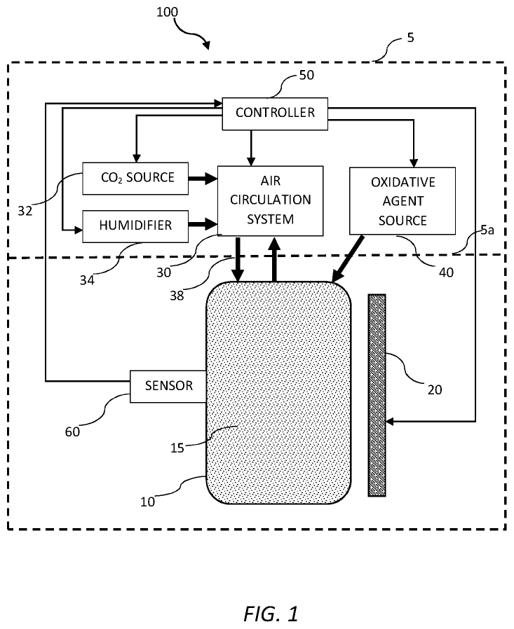

Reference is now made to FIG. 1 which is a diagrammatic illustration of a device 100 for storing live algae according to some embodiments of the invention. Device 100, according to some embodiments of the invention, may include: a closed container 10, at least one light source 20, an air circulation system 30, a CO₂ source 32 and a controller 50. In some embodiments, device 100 may further include a humidifier 34, an oxidative agent source 50 and/or a sensor 60. In some embodiments, live algae aquaculture 15 may be stored in device 100 for a period extending 4 weeks, for example, 2 months, 6 months, 12 months or more. In some embodiments, live algae aquaculture 15 may include any type of algae, (e.g., microalgae and macroalgae), for example, Nannochloropsis, Tetraselmis, Isochrysis, Pavlova, Thalassiosira weissfiogii , Thalassiosira Pseudonana, Spirulina, Chlorella and Chaetoceros.

In some embodiments, closed container 10 may be at least partially transparent to light, for example, closed container 10 may be made from a transparent polymer or glass or may include at least one window made from the transparent polymer or the glass. In some embodiments, closed container 10 may be configured to hold live algae aquaculture 15 at a predetermined temperature, for example, 4° C. In some embodiments, closed container 10 may have a volume of 0.5-20 liter. In some embodiments, closed container 10 may have a volume higher than 100 ml and lower than 50 liters.

In some embodiments, device 100 may be configured to be placed for storing in a controlled temperature chamber 5 or 5a, for example, a refrigerator cooled for 4° C. In some embodiments, device 100 may include controlled temperature chamber 5 adapted to hold the closed container. In some embodiments, controller 50 may be configured to adjust the predetermined temperature. For example, controller 50 may control the temperature in controlled temperature chamber 5 or 5a to keep the algae aquaculture alive but inhibits reproduction of the algae. The controlled temperature may be determined according to the type of the algae, for example, algae of a Nannochloropsis may be better stored at 4-8° C. and algae of Spirulina is better stored at 8-10° C.

In some embodiments, at least one light source 20 may allow providing light to closed container 10, for example, via one or more transparent walls of closed container 10 or the at least one transparent window. At least one light source 20 may include any lamp that may be configured to provide light in wavelength suitable for growing algae. For example, at least one light source 20 may include a LED array of red, yellow, green, and blue light. In some embodiments, the intensity of the light and the lightening duration of at least one light source 20 may be controlled by controller 50, to provide light to closed container 10 in sufficient intensity and sufficient duration to keep algae aquaculture 15 alive but inhibit reproduction of the algae, for example, for at least 4 weeks. For example, 10-1000 μE/m²/sec of photosynthetically active radiation (PAR) may be provided to container 10 by at least one light source 20, for up to 8 hours (e.g., 5, 6, 7 hours) every day for as long as algae aquaculture 15 is stored in closed container 10 (e.g., one month, 6 months, 12 months and more). While light source 20 is illustrated in FIG. 1 to be external to closed container 10, it should be appreciated that light source 20 may be internal to container 10. It should be further appreciated that more than one light source 20 may be used and that each of light sources 20 may be positioned either internal or external to container 10.

In some embodiments, the intensity (e.g., PAR) and duration of light provided to the algae in container 10 may be determined based on at least one of: the type of live algae, the size of closed container 10 and a required live algae density In some embodiments, there may be a direct relationship between the volume of the algae aquaculture, the density of the algae, and the amount of light to be provided. This may be due to the “path” the light travels in algae aquaculture 15. Accordingly, if containers 10 at various volumes (e.g., 1, 5, and 10 liter) may all have the same cross section dimensions (e.g., one dimension of a rectangular tank may be 10 cm) the amount of PAR to be provided (if light source 20 is located perpendicular to the side having the 10 cm dimension) may be the same. In such case, the amount of PAR provided to all containers may be 50-200 μE/m²/sec. The exact amount may be determined according to the type of algae and the required algae density. For example, the algae aquaculture that includes 10 gr/l of microalgae may require PAR of 50 μE/m²/sec while the aquaculture that include 200 gr/l of microalgae may require PAR of 200 μE/m²/sec.

In some embodiments, air circulation system 30 may allow circulating air inside closed container 10. Air circulation system 30 may include an air pump (not shown) and a pipe line 38 for providing the air to container 10. In some embodiments, the air capacity provided may be determined according to the size and volume of closed container 10, for example, a 1 liter container may be provided with 0.5-2 l/min of air, a 5 liter container may be provided with 2.5-7.5 l/min of air and a 10 liter container may be provided with 5-15 l/min of air. In some embodiments, container 10 may be provided with a valve for releasing the excess air that may be accumulated at the upper portion of container 10.

In some embodiments, humidity may be provided to the circulated air using humidifier 34. In some embodiments, air circulated in algae aquaculture 15 held in container 10 may dehydrate the aquaculture, thus may reduce the amount of water in algae aquaculture 15 and harm the algae. Accordingly, in order to reduce or eliminate the dehydration of the algae aquaculture 15 humidity may be added to the circulated air. Humidifier 34 may include a vessel that may include water, air introduced into humidifier may be bubbled and mixed with the water to gain humidity before exiting humidifier 34 and entering closed container 10. In some embodiments, the air entering the vessel must be saturated to avoid any evaporation of water from the vessel over time.

In some embodiments, CO₂ may be provided to algae aquaculture 15 in closed container 10 by CO₂ source 32. CO₂ may provide the nutrition for algae aquaculture 15 to preform photosynthesis in the presence of light. In some embodiments, CO₂ source 32 may be any reservoir or tank (e.g., a pressurized balloon or a supply line) for providing CO₂. In some embodiments the CO₂ may be provided directly to closed container 10. Additionally, or alternatively, CO₂ may be provided to the air circulating in closed container 10 (as illustrated). In some embodiments, CO₂ may be provided either before or after providing humidity to the air.

In some embodiments, a constant capacity of CO₂ may be continuously provided from CO₂ source 32 to container 10. For example, 1 liter of algae may be provided with 0.5-2 liter/min air enriched with 0.5-5 volume % of CO₂. In some embodiments, a controlled amount of CO₂ may be provided to container 10, for example, only when at least one light source 20 is illuminating Accordingly, controller 50 may control the capacity and/or duration at which the CO₂ may be provided to closed container 10. In some embodiments, the CO₂ may be provided in an amount sufficient to keep the algae aquaculture alive but insufficient to allow reproduction and thus inhibit reproduction of the algae. The amount of CO₂ provided may be determined based on at least one of: the type of live algae, the size of the closed container and a required live algae density.

In some embodiments, device 100 may include one or more sensors 60, for example, a pH sensor, a thermometer, a flow meter and the like. One or more sensors 60 may be configured to sense one or more conditions inside closed container 10. In some embodiments, controller 50 may reactive the readings from one or more sensors 60 and may control one or more controllable components of the device based on the sensed one or more conditions. For example, sensor 60 may be a pH sensor and controller 50 may control the provision of CO₂ from CO₂ source 32 according to pH level measured in algae aquaculture 15 held in container 10. Controller 50 may increase the amount (e.g., capacity and/or duration) of CO₂ when the pH level measured is below the minimum set pH level. In another example, sensor 60 may be a thermometer and controller 50 may control the temperature in controlled temperature chamber 5 according to a temperature of algae aquaculture 15 measured by sensor 60. In some embodiments, one or more sensors 60 may be located in proximity to container 10 and may measure one or more ambient conditions, for example, the ambient temperature and humidity.

In some embodiments, to prevent accumulation of undesired microorganisms (e.g., bacteria) within algae aquaculture 15 held in container 10 antiseptic agent, for example, oxidative agent may be provided periodically to algae aquaculture 15 during the entire storing time. Oxidative agent source 40 may provide the oxidative agent to container 10 according to a predetermined regime stored in a memory associated with controller 50 or may be provided in response to readings received from sensor 60. In some embodiments, the timing and amount of oxidative agent provided to closed container 10 from oxidative agent source 40 may be sufficient to prevent a grow of undesired microorganisms while not harming the live algae aquaculture. For example, a dosage of 0.5-5 mg of free chlorine may be provided for each liter of algae aquaculture 15 every day during the dark period (when light source 20 is turned off).

In some embodiments, controller 50 may include a processor (e.g., a chip) and a memory for storing thereon instructions to execute methods according to some embodiments of the invention. The memory may include instructions to provide controlled amounts of light, controlled amounts CO₂, controlled amounts oxidative agent and/or controlled temperature to container 10.

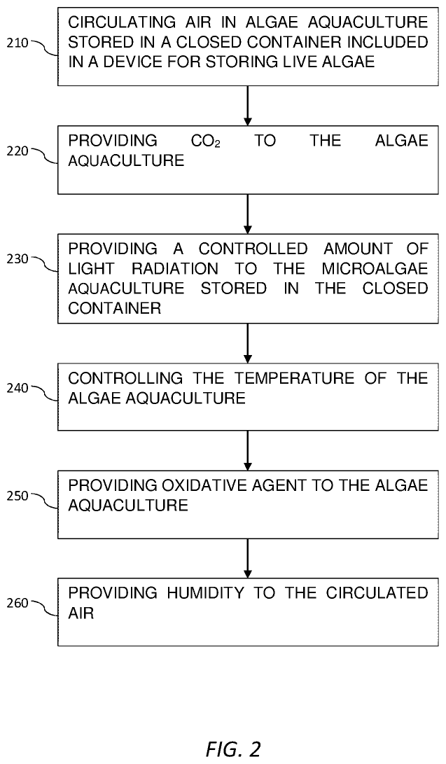

Reference is now made to FIG. 2 which is a flowchart of a method of controlling the population of live algae in a device for storing live algae according to some embodiments of the invention. Embodiments of a method, such as the one illustrated in FIG. 2, may be performed by device 100 (in FIG. 1), for example, under the control of controller 50. In box 210, air may be circulated in algae aquaculture stored in a closed container included in the device. For example, air provided by air circulation system 30 may be circulated in algae aquaculture 15 stored in closed container 10 included in device 100. In box 220, CO₂ may be provided to the algae aquaculture. For example, the CO₂ may be provided to closed container 10 from CO₂ source 23 in an amount sufficient to keep the algae aquaculture alive but that inhibits reproduction of the algae. The CO₂ may be provided either directly to container 10 or to the circulated air.

In box 230, a controlled amount of light radiation may be provided to the algae aquaculture stored in the closed container. For example, at least one light source 20 may provide light to algae aquaculture 15 in amounts sufficient to keep the algae aquaculture alive while inhibiting reproduction of the algae in the aquaculture for more than 4 weeks.

In some embodiments, the temperature of the algae aquaculture may be controlled to keep the algae aquaculture alive while reducing/inhibiting the reproduction of the algae in the aquaculture, in box 240. In some embodiments, oxidative agent may be provided to the algae aquaculture, in box 250. In some embodiments, the oxidative agent may be provided at predetermined doses at predetermined timing. As illustrated in box 260, humidity may be provided to the circulated air. In some embodiments, the amount of humidity in the air may be determined such that an amount of water in the live algae aquaculture stored in the closed container is kept substantially constant.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A device for storing live algae, comprising:
a closed container, at least partially transparent to light, the container is configured to hold live algae aquaculture at a predetermined temperature;
at least one light source for providing light to the closed container;
a CO₂ source for providing CO₂ to the closed container;
an air circulation system for circulating air inside the closed container; and
a controller for controlling the at least one light source to provide light to the closed container in an amount sufficient to keep the algae aquaculture alive but inhibits reproduction of the algae for at least 4 weeks.

2. The device of claim 1, wherein the controller is further configured to control the CO₂ source to provide CO₂ in an amount sufficient to keep the algae aquaculture alive but inhibits reproduction of the algae.

3. The device of claim 1, wherein the intensity and duration of light are determined based on at least one of: the type of the live algae, the size of the closed container and a required live algae density.

4. The device of claim 2, wherein the amount of CO₂ is determined based on at least one of: the type of the live algae, the size of the closed container and a required live algae density.

5. The device of claim 1, wherein the controller is configured to adjust the predetermined temperature.

6. The device of claim 5, further comprising a controlled temperature chamber adapted to hold the closed container.

7. The device of claim 1, further comprising at least one sensor for sensing one or more conditions inside the closed container and wherein the controller is configured to control one or more controllable components of the device based on the sensed one or more conditions.

8. The device of claim 1, further comprising: an oxidative agent source.

9. The device of claim 8, wherein the controller is further configured to control the timing and amount of oxidative agent provided to the closed container from the oxidative agent source to be sufficient to prevent a grow of undesired microorganisms while not harming the live algae aquaculture.

10. The device of claim 1, further comprising: a humidifier for adding humidity to the circulated air.

11. The device of claim 1, wherein the closed container has a volume of 0.5-20 liters.

12. A method of controlling the population of live algae in a device for storing live algae, comprising:
circulating air in algae aquaculture stored in a closed container included in the device;
providing CO₂ to the algae aquaculture; and
providing a controlled amount of light radiation to the algae aquaculture stored in the closed container,
wherein the light is provided in amounts sufficient to keep the algae aquaculture alive while inhibiting reproduction of the algae in the aquaculture for more than 4 weeks.

13. The method of claim 12, further comprising:
providing to the algae aquaculture a controlled amount of CO₂ sufficient to keep the algae aquaculture alive while inhibiting reproduction of the algae in the aquaculture.

14. The method of claim 12, further comprising:
controlling the temperature of the algae aquaculture to keep the algae aquaculture alive while inhibiting the reproduction of the algae in the aquaculture.

15. The method of claim 12, further comprising:
providing oxidative agent to the algae aquaculture.

16. The method of claim 15, wherein providing the oxidative agent comprises providing predetermined doses of oxidative agent at predetermined timing.

17. The method of claim 12, comprising:
providing humidity to the circulated air.

18. The method of claim 17, wherein the amount of humidity in the air is determined such that an amount of water in the algae aquaculture stored in the smart container is kept substantially constant.
